(19) United States
(12) Patent Application Publication
(10) Pub. No.: US 2016/0081651 A1
(43) Pub. Date: Mar. 24, 2016

(54) APPARATUS AND METHOD FOR ESTIMATING BREATHING RATE USING MICROPHONE

(71) Applicant: SOONCHUNHYANG UNIVERSITY INDUSTRY ACADEMY COOPERATION FOUNDATION, Chungcheongnam-do (KR)

(72) Inventors: Yunyoung NAM, Chungcheongnam-do (KR); Bersain A. Reyes, Coventry, CT (US); Ki H. Chon, Worcester, MA (US)

(21) Appl. No.: 14/702,778

(22) Filed: May 4, 2015

(30) Foreign Application Priority Data
Sep. 22, 2014 (KR) 10-2014-0125597

(51) Int. Cl. A61B 7/00; A61B 5/00; A61B 5/08

(52) U.S. Cl. CPC A61B 7/003; A61B 5/0816; A61B 5/7203; A61B 5/7278

(57) ABSTRACT

Provided are an apparatus and method for estimating a breathing rate using a microphone, which performs band filtering and noise filtering on tracheal and nasal sounds collected from the microphone, selects a processing region of preprocessed data, calculates similarity between pieces of data using an autocorrelation function, calculates a power spectrum density, detects multiple peaks including a highest peak through the power spectrum density, analyzes the multiple peaks to determine a breathing pattern, and calculates a breathing rate in consideration of a dynamic characteristic between inhalation and exhalation, nasal congestion detection, and noise reduction. Accordingly, it is possible to accurately estimate the breathing rate because calculation is performed in consideration of a dynamic characteristic of inhalation and exhalation, nasal congestion, and white noise.

### CROSS-REFERENCE TO RELATED APPLICATION

This application claims priority to and the benefit of Korean Patent Application No. 10-2014-0125597, filed on Sep. 22, 2014, the disclosure of which is incorporated herein by reference in its entirety.

### BACKGROUND

The present invention relates to a method of estimating a breathing rate, and more particularly, to an apparatus and method for estimating a breathing rate using a microphone, in which the breathing rate may be accurately estimated using a microphone included in a smartphone.

Breathing is one of important indices of a sign of life. In studies on 14,000 or more cardiopulmonary arrest patients, it can be seen that 44% of the patients are congenital. Thus, breathing rates of patients with a respiratory disorder may need to be continuously monitored.

The most common method of measuring a breathing rate is to passively calculate the number of breaths by viewing movement of a chest or listening to breathing sounds through a stethoscope. However, such a passive method is temporary and thus has a limitation in providing reliable data for treating patients. Accordingly, in order to enhance the reliability of the breathing rate, automation of the measurement of the breathing rate may be needed.

Recently, a sensor for measuring airflow may have been used in clinical treatment. In general, the airflow is measured by a spirometer, and the most widely used examples of the sensor include a pneumotachograph, a nasal cannulae that is connected to a pressure transducer, a heating thermistor, or a wind speed measurement device, and the like. In addition, the airflow may be measured by detecting movement of any one of a chest and a belly using a breathing inductance plethysmography (RIP), a strain gauge, or a magnetometer.

However, such a spirometer can provide accurate estimation of a breathing rate, but increases airway obstruction in addition to providing uneasy breath because breath should be taken through a mouthpiece or a face mask that is connected to a pneumotachograph. Furthermore, the spirometer requires high costs for the device itself and the use thereof, a patient should endure the discomfort whenever using the apparatus, and it is difficult to move the apparatus. An apparatus that is simple, cost-efficient, and movable and a method thereof have been required to measure the breathing rate.

In order to reflect these requirements, a solution of measuring a breathing rate using a smartphone has ever been proposed. That is, the use of the smartphone may satisfy a criterion for easy approach for estimation of the breathing rate and a criterion for economical on-demand monitoring. Recently, as a method of accurately estimating a breathing rate in a sleep state, the breathing rate may be directly obtained through a pulse stream in a finger that is captured using a camera built in a smartphone. However, it is known that the accuracy of the estimation of the breathing rate decreases when the breathing rate is 30 or more breaths per minute.

### SUMMARY

The present invention is directed to providing an apparatus and method for estimating a breathing rate using a microphone, which may accurately estimate the breathing rate using a microphone built in a smartphone or an earpiece microphone.

According to an aspect of the present invention, there is provided an apparatus for estimating a breathing rate using a microphone, including: a preprocessing unit configured to perform band filtering and noise filtering on a tracheal sound and a nasal sound that are collected from the microphone; a data selection unit configured to select a processing region of the preprocessed data; a similarity calculation unit configured to calculate similarity between pieces of data using an autocorrelation function; a power spectrum calculation unit configured to calculate a power spectrum density; a peak detection unit configured to detect multiple peaks including a highest peak through the power spectrum density; a pattern determination unit configured to analyze the multiple peaks to determine a breathing pattern; and a breathing rate calculation unit configured to calculate a breathing rate in consideration of a dynamic characteristic between inhalation and exhalation, nasal congestion detection, and noise reduction.

The breathing rate calculation unit may include: a nasal congestion detection unit configured to detect nasal congestion; and a noise detection unit configured to detect background and voice noises.

According to another aspect of the present invention, there is provided a method of estimating a breathing rate using a microphone, including: performing band filtering and noise filtering on tracheal and nasal sounds collected from the microphone; selecting a processing region of preprocessed data; calculating similarity between pieces of data using an autocorrelation function; calculating a power spectrum density; detecting multiple peaks including a highest peak through the power spectrum density; analyzing the multiple peaks to determine a breathing pattern; and calculating a breathing rate in consideration of a dynamic characteristic between inhalation and exhalation, nasal congestion detection, and noise reduction.

The calculation of the breathing rate may be performed using any one of a Welch periodogram method, an AR power spectrum (Burg algorithm), and a modified covariance method.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a block diagram showing a control circuit of a breathing rate estimation apparatus using a microphone according to an embodiment of the present invention;

FIG. 2 is a flowchart showing a method of estimating a breathing rate using a microphone according to an embodiment of the present invention;

FIGS. 3A-3C illustrate sample graphs of a tracheal breath sound and a nasal breath sound and an analysis diagram;

FIGS. 4A-4D illustrate graphs of raw data and filtered data of a tracheal sound and a nasal sound;

FIGS. 5A-5D illustrate sample graphs of a PSD using a sound envelope and an AR model;

FIGS. 6A-6F illustrate peak graphs of a PSD using a sound envelope and an AR model;

FIGS. 7A-7D illustrate graphs of median and interquartile range (IQR) errors that are measured from a breathing rate result calculated by a maximum peak in a PSD using a sound envelope and an AR model;

FIGS. 8A-8C illustrate graphs of median and IQR errors that are measured from a breathing rate result calculated by a maximum peak or by the maximum peak and a second maximum peak in a PSD using an sound envelope and an AR model;

FIGS. 9A-9D illustrate graphs showing a typical nasal sound signal, sound spectrum, amplitude spectrum, and PSD that are obtained by a microphone built in a smartphone;

FIGS. 10A-10C illustrate raw data of a nasal breath sound that is recorded while breathing naturally when a distance between a nasal cavity and a smartphone is 30 cm; and

FIGS. 11A-11C illustrate a sample of a nasal breath sound that is recorded along with a background voice noise while breathing naturally when a distance between a nasal cavity and a smartphone is 30 cm.

### DETAILED DESCRIPTION

Exemplary embodiments of the present invention will be described in detail below with reference to the accompanying drawings. While the present invention is shown and described in connection with exemplary embodiments thereof, it will be apparent to those skilled in the art that various modifications can be made without departing from the spirit and scope of the invention.

The present invention proposes a new method of estimating a breathing rate using a nasal breathing sound record in a smartphone. The method detects a nasal airflow using a microphone built in a smartphone and an earpiece microphone. In this case, in order for comparison of experimental results, an actual breathing rate is measured by installing a breathing belt around a chest and a belly of an experimental subject. Meanwhile, a tracheal breath sound and a nasal breath sound are recorded using the built-in microphone disposed around a larynx and the earpiece microphone attached to a philtrum disposed under a nasal cavity. Inhalation and exhalation are detected by average power of the nasal breath sound. The breathing rate is estimated using different calculation methods (applying a sound envelope and an autoregressive (AR) model). In order to enhance the accuracy of estimating the breathing rate, a breathing pattern is determined by a plurality of dominant peaks in a power spectrum density (PSD). In particular, since a frequency spectrum of inhalation and exhalation is different depending on personal characteristics including a nasal congestion and a dynamic difference between the inhalation and exhalation, the detection of nasal congestion and the reduction of white noise are considered. In order to evaluate a performance of the present invention, data was collected from 10 healthy experimental subjects (normal persons). In a breathing range (12-90 breaths/minute), a result of the experiment according to an embodiment of the present invention may show a great enhancement in performance, compared to existing methods that use average power of tracheal breath sound signals. This may provide convenience for patients and also save time and money. As a result, the present invention may be easily used to analyze and diagnose a patient with a respiratory disorder. This may result from a powerful data analysis tool of a mobile device having a microphone.

Meanwhile, a stethoscope is a device that is commonly used by a doctor to determine a physical condition of a respiratory system. Given that the stethoscope is basically a kind of microphone, it is not surprising that the breathing rate is obtained using a microphone. There are several methods of determining the breathing rate using the stethoscope. In this case, in order to determine an accurate breathing rate, an inhalation sound signal and an exhalation sound signal should be distinguished from each other. Fortunately, since dynamics of the inhalation and exhalation are different, two phases of the inhalation and exhalation can be definitely identified using multiple different approaches. Well-known automated approaches for estimating the breathing rate includes change in strength of a breathing sound, relative change in total sound power, analysis of tracheal sound entropy, and analysis of biological sound. The breathing sound may be obtained by positioning a microphone on a carotid artery of a neck or a nasal cavity itself. An exhalation sound recorded in a trachea is a little louder than, but has a characteristic similar to, an inhalation sound. On the contrary, intensities of the nasal breath sounds upon inhalation and exhalation that are recorded around a nasal cavity of an experimental subject are definitely different from each other. Accordingly, the present invention proposes a method of utilizing sound characteristics of a breath measured in any one of the trachea and the nasal cavity, and estimating an accurate breathing rate of a wide range of a replayable sound signal using a built-in microphone and a microphone of a headset connected to a smartphone though a cable. The present invention provides a method of reliably determining a breathing rate from any one of the trachea and the nasal cavity only using a built-in microphone or an ear-microphone of the smartphone.

An apparatus and method for estimating a breathing rate using a microphone according to an embodiment of the present invention will be described below with reference to the accompanying drawings.

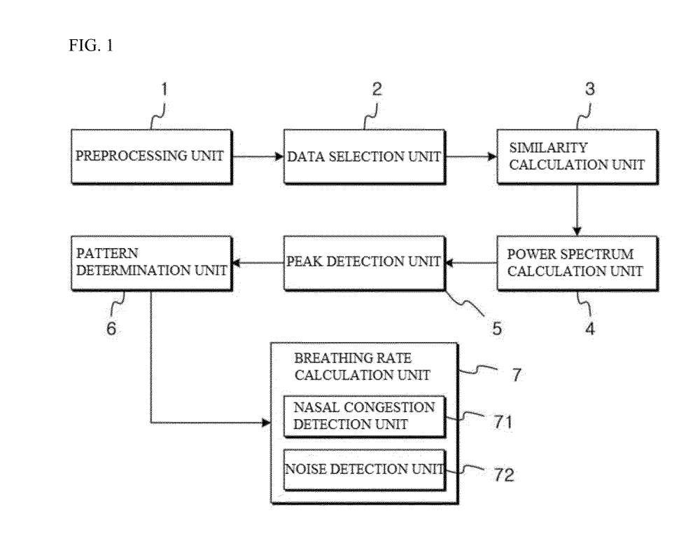

FIG. 1 is a block diagram showing a control circuit of a breathing rate estimation apparatus using a microphone according to an embodiment of the present invention.

Referring to FIG. 1, the breathing rate estimation apparatus using a microphone according to an embodiment of the present invention includes a preprocessing unit 1 including a band-pass filter and a spline filter, a data selection unit 2 configured to select a data region to be processed, a similarity calculation unit 3 configured to calculate similarity between pieces of data using an autocorrelation function, a power spectrum calculation unit 4 configured to calculate a power spectral density, a peak detection unit 5 configured to detect multiple peaks including a highest peak through the power spectral density, a pattern determination unit 6 configured to analyze the multiple peaks to determine a breathing pattern, and a breathing rate calculation unit 7 configured to calculate a breathing rate in consideration of a dynamic characteristic between inhalation and exhalation, nasal congestion detection, and noise reduction.

Here, the breathing rate calculation unit 7 includes a nasal congestion detection unit 71 configured to detect a nasal congestion and a noise detection unit 72 configured to detect background and voice noises.

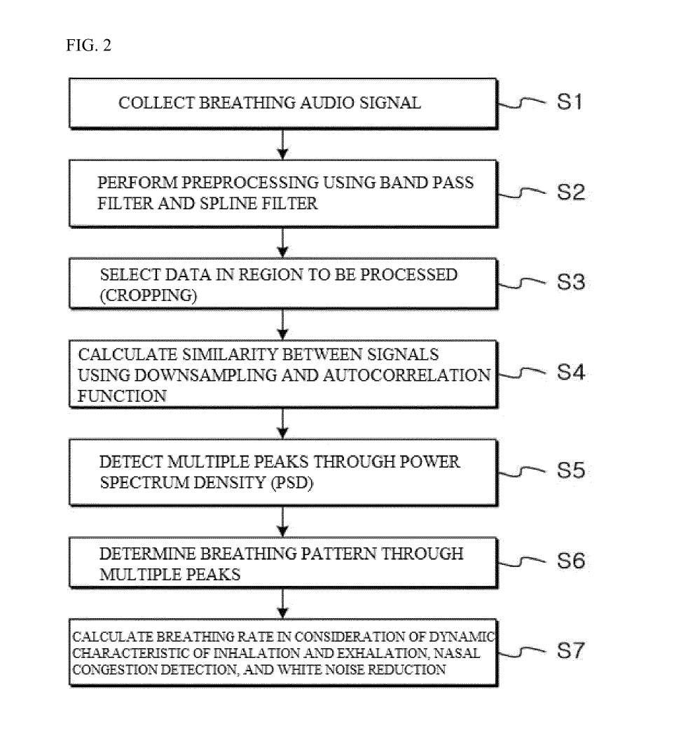

FIG. 2 is a flowchart showing a method of estimating a breathing rate using a microphone according to an embodiment of the present invention.

Referring to FIG. 2, the method of estimating the breathing rate using the microphone according to an embodiment of the present invention includes collecting a breathing audio signal through a microphone corresponding to breathing sounds generated from the trachea and the nasal cavity (S1) and then preprocessing a digitalized breathing audio signal first (S2). That is, the breathing audio signal is fed through a low pass filter (LPF) of 0.19 Hz and a high pass filter (HPF) of 4.6 Hz (Band Pass Filter). A digital audio sampling rate of a smartphone is 44.1 kHz, but 100 Hz is sufficient to calculate the breathing rate in real time. However, when the sampling rate is less than 100 Hz, a signal value is supplemented to 100 Hz using a cubic spline algorithm (Spline Filter).

Next, the first part and the last part of a processed signal, for example, the first 10 seconds and the last 10 seconds are not used to process data for calculating the breathing rate (Cropping) (S3).

The method includes downsampling data that is obtained to perform real-time processing and enhance a calculation speed from 100 Hz to 10 Hz (Downsampling) and calculating a similarity between signals using an autocorrelation function (Autocorrelation) (S4).

Such a signal is used to detect multiple peaks including a highest peak through a power spectrum density (PSD) (Peak Detection) (S5).

Before calculating the breathing rate, the method includes analyzing breathing characteristics through the multiple peaks to determine a breathing pattern (Breathing Pattern Determination) (S6).

The method includes detecting nasal congestion and noise according to dynamic characteristics and personal characteristics between inhalation and exhalation and calculating the breathing rate finally (Breathing Rate Calculation) (S7).

Here, examples of a method that is used to calculate the breathing rate include a Welch periodogram method, an AR power spectrum (Burg algorithm), and a modified covariance method. The PSD may be calculated using the Welch periodogram method.

Data Collection

Data is collected while a healthy experimental subject sits up straight. Tracheal and nasal breathing sound signals are recorded using a microphone built in a smartphone and an ear microphone. In this case, the microphones are positioned on a suprasternal notch of a neck, and a philtrum under a nasal cavity of the experimental subject. In order to assume that the microphone is fixed while measuring the nasal breathing sound, the measurement is performed along with a microphone of an earphone that is positioned around the nasal cavity of the experimental subject. To determine an actual breathing rate, an impedance-based chest belt sensor is installed on a chest and a belly of the experimental subject.

While microphone data is directly collected in a smartphone at a digitalized sampling speed of 100 Hz, an electrocardiogram (ECG) signal and an impedance-based chest belt sensing signal are used to obtain data using Labchart software (AD Instruments) at a sampling rate of 400 Hz. In order to test the reliability and the accuracy of a program, the estimated breathing rate is compared with the actual breathing rate acquired from the breathing impedance belt signal. In particular, the average intensities of the inhalation and the exhalation between the trachea and the nasal cavity of the experimental subject sitting up straight are used to derive estimation of the breathing rate.

The data is collected from 10 healthy non-smokers, aged in 20 to 40s. All experimental subjects breathe according to a signal sound having a predetermined time length and programmed at a metronome speed at a selected frequency. Each experimental subject exhales before generating a next signal sound and inhales at each signal sound. Data is collected in a breathing frequency range of 0.2 Hz to 1.5 Hz while the breathing frequency is increased by 0.1 Hz. At a metronome frequency programmed for each experimental subject, nasal breath data (with the mouth being close) is collected during 3 minutes.

Preprocessing

An audio file recorded as a mono audio WAVE file of 44,100 Hz and 16 bits is low-pass filtered with a cut-off frequency of 5 kHz. Such sound signals are digitalized at a rate of 100 Hz. The audio signals are deliberately digitalized at a low rate in order to reduce a calculation time and a data capacity. This is given by reflecting the fact that a highest breathing rate is 2 Hz at most. Sound level meter application software of a smartphone may provide a linear audio scale in the range of 0 to 110 dB. In this experiment, the audio signal is observed in the range of 40 to 105 dB. The experiment is conducted in a silent room, and a sound generated by a ceiling fan has the same level as a background noise of about 40 dB.

As shown in FIG. 2, a process of preprocessing the digitalized audio signal includes band pass filtering (0.19 Hz and 4.6 Hz) caused by downsampling data from 100 Hz to 10 Hz. In all methods for removing a background sound noise from the breathing sound, a first step is to focus segments. In order to effectively remove a background sound from calculated tracheal or nasal sound energy, a segment including the background sound is localized, and the tracheal or nasal sound energy (a difference between nasal sounds in a time domain) is calculated with the segment including the background sound being ignored. Next, spline interpolation is applied to estimate a value of the tracheal or nasal sound energy from a segment in which an influence of the background sound is effectively cancelled in the estimated energy of the tracheal or nasal sound.

Data Analysis

In order to extract features of experimental data, a sample is repeated by 3,072 between continuous windows at a sampling frequency of 100 Hz, and a window size is set to 6,144. Inhalation and exhalation are detected by average power of the tracheal and nasal sounds. Both of the tracheal and nasal sound signals are divided into 6,144 samples. Autocorrelation of a detrended nasal sound signal is calculated and windowed by a hamming window. A power spectrum is calculated by a fast Fourier transform (FFT) of the windowed autocorrelation. In order to find an appropriate breathing phase, the band pass amplitude in which the tracheal and nasal sounds are filtered is examined using three different methods (a Welch periodogram method, an autoregressive (AR) power spectrum analysis technique (Burg algorithm), and a modified covariance method of linear prediction). First, a PSD of each segment is calculated using the Welch periodogram method. The square magnitude of the Fourier transformation is generally referred to as a periodigram, which is an estimator of a power spectrum density. There is no consistent estimator because an individual value does not tend to be limited to a sample size that increases exponentially. Second, a general PSD estimator reduces autocovariance to reach a spectrum window having a certain width. This allows low sampling diversity and enables consistent estimation only through a few assumptions. In general, mostly, the actual breathing rate is found by calculating a PSD of a breath tracking signal and finding a frequency at a maximum amplitude. However, sometimes, the breathing rate cannot be measured only by the Fourier transformation of the autocorrelation function and the Welch periodogram method.

The AR power spectrum analysis technique is also used to analyze fluctuation of detrended time series. The AR power spectrum analysis technique is based on a recursive least square algorithm that makes a regression identification procedure appropriate to update coefficients of all new periodic models. In past studies, an autoregressive power spectrum analysis was used to examine an interval of the breathing rate and a change in blood pressure (BP). Likewise, a frequency of a bit-to-bit change in the breath may be estimated by the autoregressive power spectrum analysis. According to an embodiment of the present invention, regressive model orders that are used to estimate a PSD for the AR and generate a length of a discrete Fourier transform (DFT) are set to 50 and 256, respectively. Furthermore, the modified covariance method of linear prediction is also used to extract a frequency in the maximum amplitude by using a least square technique for estimating a linear prediction coefficient from data that is sampled by simultaneous minimization of front and back linear prediction, which is an error square.

A normal nasal breath sound is a broadband spectrum having some peaks. When an amount of flow of a breath is changed into an amplitude and energy, a shape and a peak of a spectrum curve geometrically and pathologically change an upper air flow. A variety of impermanency of the nasal breath sound and an influence of a flow speed on a spectral function are examined. Main features include average power, a sound envelope, and a center frequency. A relation between the flow and the average power of the nasal sound may change a peak flow. The breathing sound is commonly a non-stop signal. To overcome this problem, in all breathing periods of inhalations and exhalations, a sound segment in which a corresponding flow rate is equal to or greater than 10% of the maximum flow in a corresponding breathing period was considered to examine the sound segment. A first feature extraction algorithm is based on some dominant or assistant or higher order peaks of the sound envelope. A second feature extraction algorithm is based on a minimum Euclidean distance between two frequency bands. A third feature extraction algorithm is based on a peak pattern in the PSD.

In sound envelope extraction, a Hilbert transform on a continuous-time signal x(t) is defined as follows.

$$H ( x ( t ) ) = \frac{1}{π} {\int }_{- ∞}^{∞} x ( τ ) \frac{1}{t - τ} d τ \qquad [ Equation 1 ]$$

The Hilbert transform is used to extract an envelope of a filtered discrete sound signal. An obtained amplitude envelope signal is smoothed and downsampled. An amplitude of the obtained smoothed envelope signal is indicated as a(m), which is a time index after downsampling. The smoothing is an essential part of an embodiment of the present invention. A procedure thereof is as follows.

1) A peak frequency of a(m) is determined from the maximum of a power spectrum (512 point FFT, MATLAB function pwelch).

2) Cubic spline interpolation is used to obtain filtered amplitude time series using a band-pass filter of 0.19 to 4.6 Hz (MATLAB function spline).

3) Here, after performing downsampling from 100 Hz to 10 Hz, an envelope amplitude of band-pass filtered a(m) is calculated as a magnitude of an analysis signal (a complicated value). The analyzed signal is generated from a sum of the band-pass filtered a(m) and the Hilbert transform (MATLAB function Hilbert).

4) A maximum value of an amplitude envelope of a(m) is determined, and an average value is calculated using a window around a peak.

5) Some a(m) includes two or more frequency components, as shown with two or more peaks in the power spectrum.

In order to determine a pattern of two or more peaks in the PSD, a peak is defined as follows.

p(i)={kᵢ, 0<i<n} [Equation 2]

where n is the number of peaks, kᵢ is an estimate of the PSD, and i is a breathing rate or an interval between inhalation and exhalation. Accordingly, it is essential to extract some dominant or second (or higher) order peaks. In order to achieve this, in an arrangement element kᵢ, returned indices that keep an original order are aligned in descending order.

Thus, a breathing pattern algorithm may be defined as follows.

1) first, kᵢ is aligned in descending order of the PSD estimation. S={A¹, A², . . . , A^{Z}} shows an aligned list.

2) m peak vertices are selected to estimate the breathing rate. The breathing pattern P is determined as follows.

P=10^{m-1}×A¹+10^{m-2}×A²+ . . . +A^{m}, 1≦m≦Z [Equation 3]

where Z is the total number of peaks. For example, when m is set to be 3, P is calculated in 6 possible combinations according to an order of peak points such as ‘123,’ ‘132,’ ‘213,’ ‘231,’ ‘312,’ and ‘321.’ The shape of the sound envelope is estimated by P in each phase. For example, when P is ‘123’ or ‘132,’ the shape of the sound envelope has an approximately asymmetric distribution. When P is not ‘123’ or ‘132,’ the shape has an asymmetric distribution, and the breathing rate may be simply calculated by performing division into two.

Basically, the breathing rate may be calculated a first-order peak A¹ of the PSD using the sound envelope and the AR model. In general, an intermediate error in estimation of the breathing rate based on the first-order peak is greater than that of a high-frequency (HF) breathing rate in any other methods. In order to improve the accuracy of the estimation of the breathing rate, a breathing frequency may be identified with a maximum peak of the power spectrum of breathing data. The breathing frequency may be determined as a frequency corresponding to the maximum peak of the PSD. However, a frequency spectrum of inhalation and exhalation is different depending on individual characteristics such as a nasal congestion and a dynamic difference between the inhalation and the exhalation.

According to the present experiment, any experimental subject suffered from a nasal congestion associated with a common cold or a rhinitis. In this case, a derived breathing rate was twice as high as in other cases. To solve this problem, when the nasal congestion is detected, the breathing rate should be calculated again. In an embodiment of the present invention, a minimum Euclidean distance of P and a probability density function are considered.

In general, a breathing rate may be measured from a derived breathing rate in which recorded inhalation and exhalation sound power is divided by two sorts that are similar to each other. Furthermore, intermediate detection errors may be observed from a low-frequency (LF) breathing rate caused by a white noise. According to the present experiment, when P is 200 or more, that is, P is one of 4 possible combinations 213, 231, 312, and 321, a reference f is as follows.

$$\begin{matrix}{d}_{min} = arg {min}_{i} \vert {A}^{i} - {A}^{i + 1} \vert & [ Equation 4 ] \\ D = \vert {A}^{1} - ω \cdot {d}_{min} \vert & [ Equation 5 ] \\ f = \{ \begin{matrix}true , & if P > ρ and D < {d}_{th} \\ false , & otherwise\end{matrix} & [ Equation 6 ]\end{matrix}$$

where ω is a weight vector, ρ is a reference for detecting a white noise, D is a distance dₘᵢₙ between a maximum peak and a minimum peak, and dₜₕ is a threshold value. According to the present experiment, ω and ρ are set to 2 and 200, respectively.

In particular, a condition for detecting an additional white noise is a simple logical AND condition that is given by the following calculation.

$$\frac{k ( {A}^{i + 2} )}{k ( {A}^{i} )} > σ AND \vert k ( {A}^{i} ) - 2 \cdot D \vert < δ \qquad [ Equation 7 ]$$

According to the present experiment, σ and δ are set to 0.1 and 0.01, respectively.

### CONCLUSION

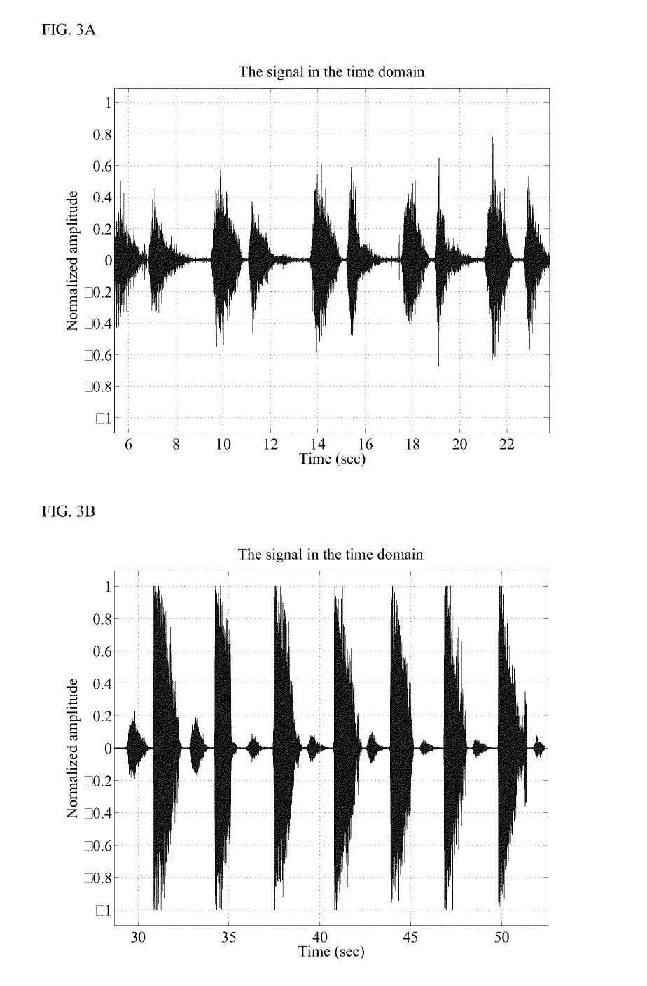

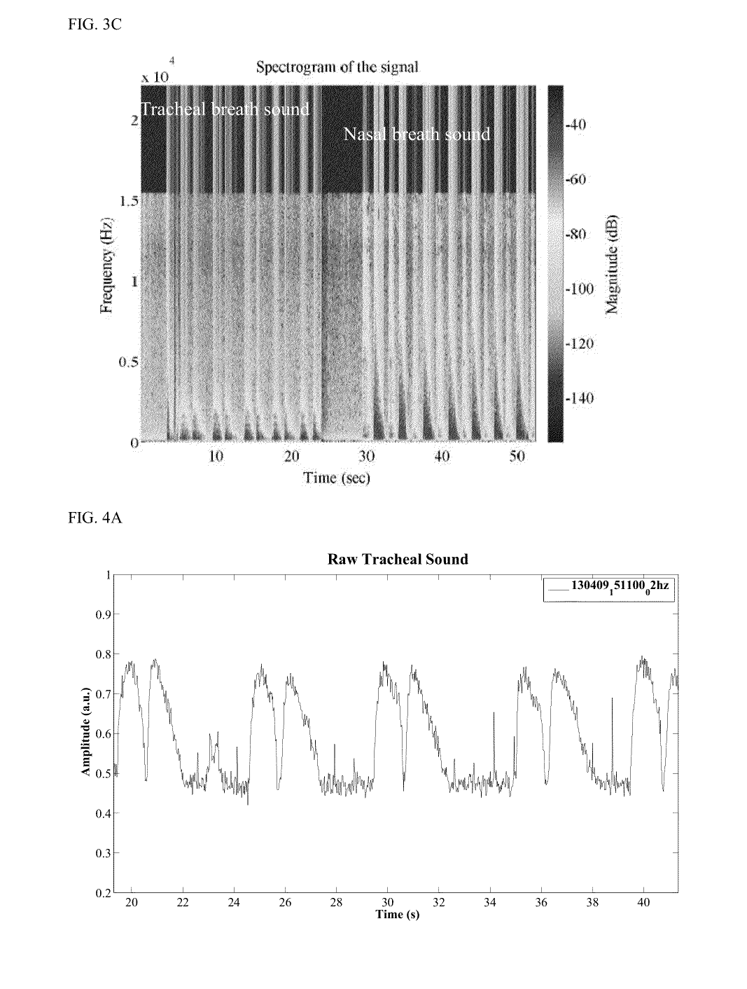

FIGS. 3A-3C illustrate sample graphs of a tracheal breath sound and a nasal breath sound and an analysis diagram.

Referring to FIGS. 3A-3C, graphs recorded by an earpiece microphone and a sound spectrum for 20 seconds are shown. FIG. 3A and FIG. 3B show raw data of a tracheal breath sound and raw data of a nasal breath sound, respectively. FIG. 3C shows a sound spectrum in which a tracheal breath signal and a nasal breath signal are observable. A vertical axis indicates a frequency distribution, and a horizontal axis indicates a time (in seconds). Each color, which changes from red to blue, represents power of a signal at a specific time and a specific frequency. Inhalation and exhalation tend to have similar characteristics in terms of frequency assignment. There is a unique difference in that it is observed that the nasal breath sound has more power than the tracheal breath sound. Power of the nasal breath sound during inhalation is less than that during exhalation.

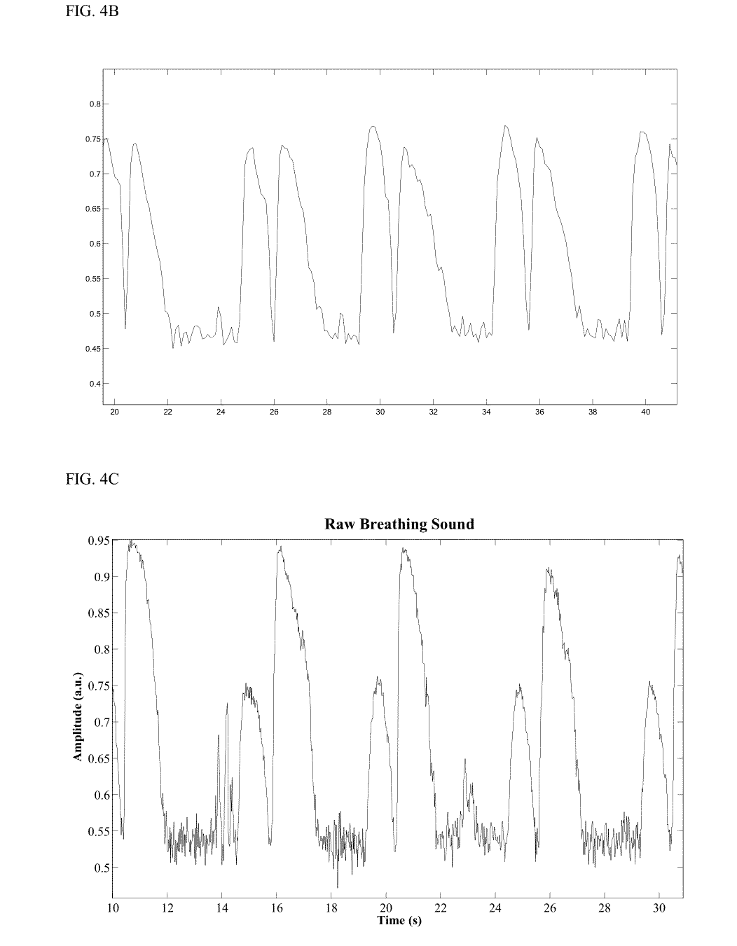

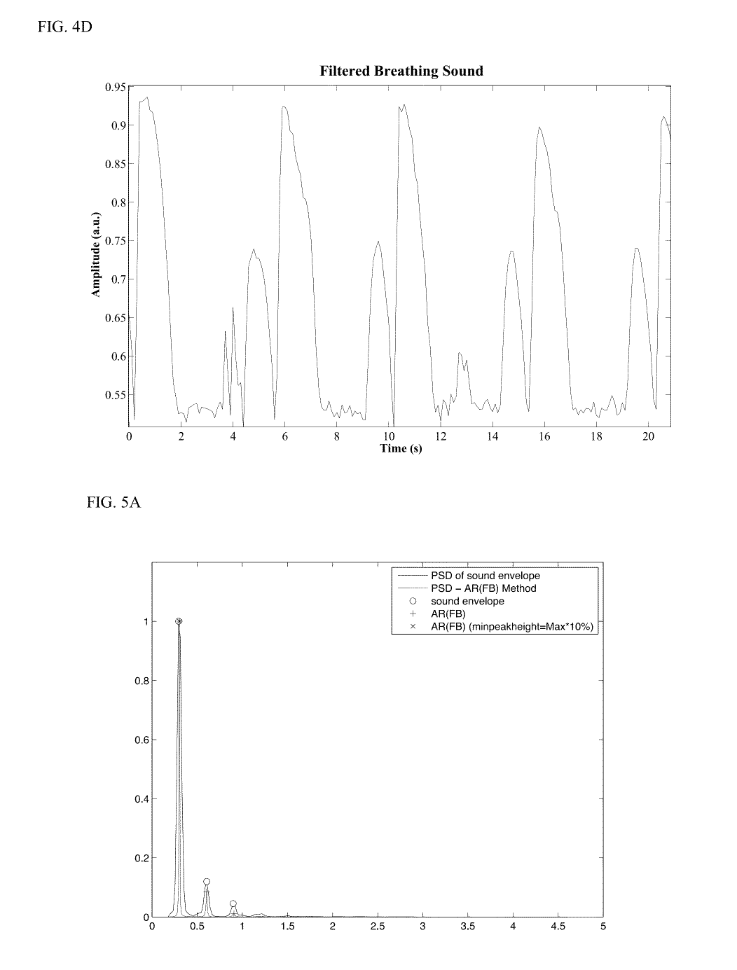

FIGS. 4A-4D illustrate graphs of raw data and filtered data of a tracheal sound and a nasal sound.

FIGS. 4A and 4C record magnitudes of a tracheal sound and a nasal sound for three minutes, from a built-in microphone and an earpiece microphone of a smartphone. As shown in FIGS. 4B and 4D, it can be seen that activities of all undesired sounds have basically been removed from raw data after cubic spline interpolation. This data of a specific experimental subject was recorded by the same apparatus. Comparing recorded raw data and a filtered signal, it can be seen that the filtered signal follows an absolute value of a flow signal. A relative flow rate estimation result from the filtered signal is associated with a closely corresponding record flow.

As a relevant flow without calibration, an estimated amplitude of the flow does not indicate an actual amount of a liter flow per second.

For each breathing frequency, a detection error for each frequency was found from all experimental subjects who used different techniques. An estimated error of the breath is calculated from each breathing frequency.

$$ε = \frac{{mean ( R - {R}_{est} )}^{2}}{{mean ( R )}^{2}} \times 100 \qquad [ Equation 8 ]$$

where R and Rₑₛₜ indicate an actual value and an estimated value of the breathing rate. An error value is an average value for all experimental subjects with respect to inhalation and exhalation phases.

Table 1 summarizes median errors and interquartile range (IQR) errors measured from a breathing rate result that is obtained from the tracheal and nasal breath sound signals in a breathing range of 0.2 to 1.5 Hz. As provided as numerical values in Table 1, median errors obtained form the breathing rate result measured from the tracheal and nasal breath sound signals are 9.741 and 0.015, respectively. In this table, the breathing rate measured from the nasal breathing sound provided the lowest error among all the breathing rates being compared. As a result, it can be seen that a breathing rate estimation technique is improved by acquiring the nasal breathing sound.

TABLE 1

| | | Tracheal sound | Nasal sound | | |
|---|---|---|---|---|---|
| | | Sound | Sound | | Sound |
| | | envelope | envelope | AR | envelope |
| Breathing | | using | using | using | using |
| rate | Error | Max peak | Max peak | Max peak | two peaks |
| 12 | Median | 5.85 ± 10.911 | 0.055 ± [symbol] 3.108 | 0.0 [symbol] 4 ± [symbol] 0.132 | 0.551 ± [symbol] .227 |
| (0.2 Hz) | IQR | 6.186 ± 13.123 | 0 ± 0 | 25.064 ± 11.824 | 0.496 ± [symbol] .234 |
| 18 | Median | 29.43 ± [symbol] 7.061 | 0.055 ± [symbol] 1.097 | 0.003 ± [symbol] 1.1 | 0.174 ± [symbol] 1.06 |
| (0.3 Hz) | IQR | 24.625 ± [symbol] 9.685 | 0 ± 0 | 0.012 ± 0.005 | 0.03 ± 0.014 |
| 24 | Median | 10.14 ± 12.288 | 0.055 ± [symbol] 0.1 | 0 ± [symbol] 3.117 | 0.064 ± [symbol] .41 |
| (0.4 Hz) | IQR | 11.802 ± [symbol] 5.036 | 0.01 ± 0.004 | 0 ± 0 | 0.01 ± 0.004 |
| 30 | Median | 24.51 ± 31.173 | 0.024 ± 29.526 | 0.002 ± [symbol] 0.707 | 0.055 ± 29.786 |
| (0.5 Hz) | IQR | 14.589 ± [symbol] 4.678 | 0 ± 0 | 0 ± 0 | 7.042 ± [symbol] .32 |
| 36 | Median | 21.44 ± [symbol] .529 | 0.006 ± [symbol] 9.148 | 0.005 ± [symbol] 9.514 | 0.055 ± [symbol] .675 |
| (0.6 Hz) | IQR | 11.421 ± 24.621 | 0.012 ± 0.005 | 24.29 ± 11.45 | 0.012 ± 0.005 |
| 42 | Median | 20.19 ± [symbol] 8.765 | 0.002 ± [symbol] 0.534 | 0.002 ± 0.031 | 0.002 ± 0.021 |
| (0.7 Hz) | IQR | 10.314 ± 25.213 | 0.026 ± 0.012 | 0.002 ± 0.001 | 0.013 ± 0.006 |
| 48 | Median | 24.15 ± [symbol] 2.63 | 0 ± [symbol] 8.67 | 0 ± [symbol] .155 | 0 ± 14.87 |
| (0.8 Hz) | IQR | 9.187 ± 19.488 | 20.444 ± [symbol] .637 | 25.107 ± 11.836 | 2.179 ± [symbol] .027 |
| 54 | Median | 16.9 ± 11.242 | 0 ± [symbol] 8.598 | 0 ± [symbol] 8.6 | 0 ± 15.403 |
| (0.9 Hz) | IQR | 11.005 ± [symbol] 3.344 | 20.731 ± [symbol] .773 | 20.701 ± [symbol] .759 | 2.334 ± 1.1 |
| 60 | Median | 19.29 ± 10.309 | 0.002 ± [symbol] 5.065 | 0.002 ± [symbol] 7.626 | 0.002 ± 12.581 |
| (1.0 Hz) | IQR | 5.844 ± 12.397 | 0 ± 0 | 21.791 ± 10.272 | 7.5 [symbol] 4 ± [symbol] .575 |
| 66 | Median | 27.67 ± [symbol] 7.185 | 0.003 ± [symbol] 0.384 | 0.001 ± 29.851 | 0.015 ± [symbol] .557 |
| (1.1 Hz) | IQR | 6.132 ± 13.007 | 0 ± 0 | 0.001 ± 0 | 0.836 ± 0.394 |
| 72 | Median | 28.88 ± [symbol] 6.002 | 0.005 ± [symbol] 0.116 | 0 ± [symbol] .117 | 0.008 ± [symbol] .029 |
| (1.2 Hz) | IQR | 3.266 ± [symbol] .928 | 0.001 ± 0 | 0 ± 0 | 0.72 ± 0.339 |
| 78 | Median | 22.67 ± [symbol] .122 | 0.004 ± 29.891 | 0 ± 29. [symbol] 92 | 0.007 ± [symbol] 6.27 |
| (1.3 Hz) | IQR | 0.018 ± 0.03 [symbol] | 0 ± 0 | 0 ± 0 | 34.031 ± [symbol] 6.057 |
| 84 | Median | 22.14 ± [symbol] .827 | 0.002 ± 29.699 | 0.001 ± 29.699 | 1.114 ± 18.204 |
| (1.4 Hz) | IQR | 0 ± 0 | 0 ± 0 | 0 ± 0 | 16.897 ± [symbol] .718 |
| 90 | Median | 22.67 ± [symbol] .104 | 0.001 ± 0 | 0.001 ± [symbol] 9.895 | 5.389 ± [symbol] 9.264 |
| (1.5 Hz) | IQR | 0.155 ± 0.331 | 0 ± 0 | 24.955 ± 11.755 | 50.429 ± 13.818 |

| | Nasal sound | | | |
|---|---|---|---|---|
| | | | ARconsidering | |
| | | AR | nasal | AR |
| | | considering | congestion | considering |
| | AR | nasal | detection & | sound |
| Breathing | using | congestion | white noise | envelope |
| rate | two peaks | detection | reduction | shape |
| 12 | 0.055 ± 0.004 | 0.064 ± [symbol] 0.132 | 0.064 ± 0.025 | 1.752 ± 2.125 |
| (0.2 Hz) | 0.01 ± 0.004 | 25.084 ± 11.624 | 0.023 ± 0.011 | 2.527 ± 1.032 |
| 18 | 0.032 ± [symbol] 7.229 | 0.008 ± [symbol] 1.1 | 0.008 ± 0.014 | 0.055 ± 27.21 |
| (0.3 Hz) | 0.171 ± 0.078 | 0.012 ± 0.005 | 0 ± 0 | 0.271 ± 0.0128 |
| 24 | 0 ± [symbol] .115 | 0 ± [symbol] 0.117 | 0 ± 0 | 0.055 ± 0.027 |
| (0.4 Hz) | 0.098 ± 0.046 | 0 ± 0 | 0 ± 0 | 0.064 ± 0.028 |
| 30 | 0.019 ± [symbol] 0.93 | 0.002 ± 0.707 | 0.002 ± 0.001 | 0.024 ± [symbol] 20.934 |
| (0.5 Hz) | 7.072 ± [symbol] .331 | 0 ± 0 | 0 ± 0 | 0.023 ± 0.011 |
| 36 | 0.008 ± 0.019 | 0.005 ± [symbol] 9.514 | 0.006 ± 0.002 | 0.008 ± 0.019 |
| (0.6 Hz) | 0.015 ± 0.006 | 24.29 ± 11.45 | 0.001 ± 0 | 0.012 ± 0.005 |
| 42 | 0.009 ± 0.016 | 0.002 ± 0.031 | 0.002 ± 0.031 | 0.002 ± 0.031 |
| (0.7 Hz) | 0.013 ± 0.005 | 0.002 ± 0.001 | 0.002 ± 0.001 | 0 ± 0 |
| 48 | 0 ± [symbol] 5.954 | 0 ± 0.005 | 0 ± 0.005 | 0 ± [symbol] 8.667 |
| (0.8 Hz) | 0.954 ± 0.449 | 0.003 ± 0.001 | 0.003 ± 0.001 | 0 ± 0 |
| 54 | 0 ± [symbol] 3.666 | 0 ± 0.061 | 0 ± 0.061 | 0 ± 0.13 [symbol] |
| (0.9 Hz) | 16.125 ± 0.449 | 0.004 ± 0.002 | 0.004 ± 0.002 | 0.014 ± 0.006 |
| 60 | 0.003 ± 7.819 | 0.002 ± 0.033 | 0.002 ± 0.033 | 0.002 ± 0.033 |
| (1.0 Hz) | 1.093 ± 0.515 | 0.002 ± 0.001 | 0.002 ± 0.001 | 0 ± 0 |
| 66 | 0.003 ± 2.728 | 0.001 ± 0.001 | 0.001 ± 0.001 | 0.003 ± 0.003 |
| (1.1 Hz) | 0.76 ± 0.358 | 0 ± 0 | 0 ± 0 | 0 ± 0 |
| 72 | 0 ± 22.923 | 0 ± 0 | 0 ± 0 | 0.005 ± 0.003 |
| (1.2 Hz) | 3.504 ± 1.652 | 0 ± 0 | 0 ± 0 | 0.005 ± 0.002 |
| 78 | 0 ± [symbol] 2.783 | 0 ± 0 | 0 ± 0 | 0.004 ± 0.001 |
| (1.3 Hz) | 68.129 ± [symbol] 2.116 | 0 ± 0 | 0 ± 0 | 0.003 ± 0.001 |
| 84 | 0.002 ± 22.339 | 0.001 ± 0 | 0.001 ± 0 | 0.002 ± 0.002 |
| (1.4 Hz) | 5.157 ± 2.431 | 0 ± 0 | 0 ± 0 | 0 ± 0 |
| 90 | 5.458 ± [symbol] 0.847 | 0.001 ± 0 | 0.001 ± 0 | 0.001 ± 0.024 |
| (1.5 Hz) | 60.661 ± 27.4 | 0± 0 | 0 ± 0 | 0 ± 0 |

[symbol]

indicates data missing or illegible when filed

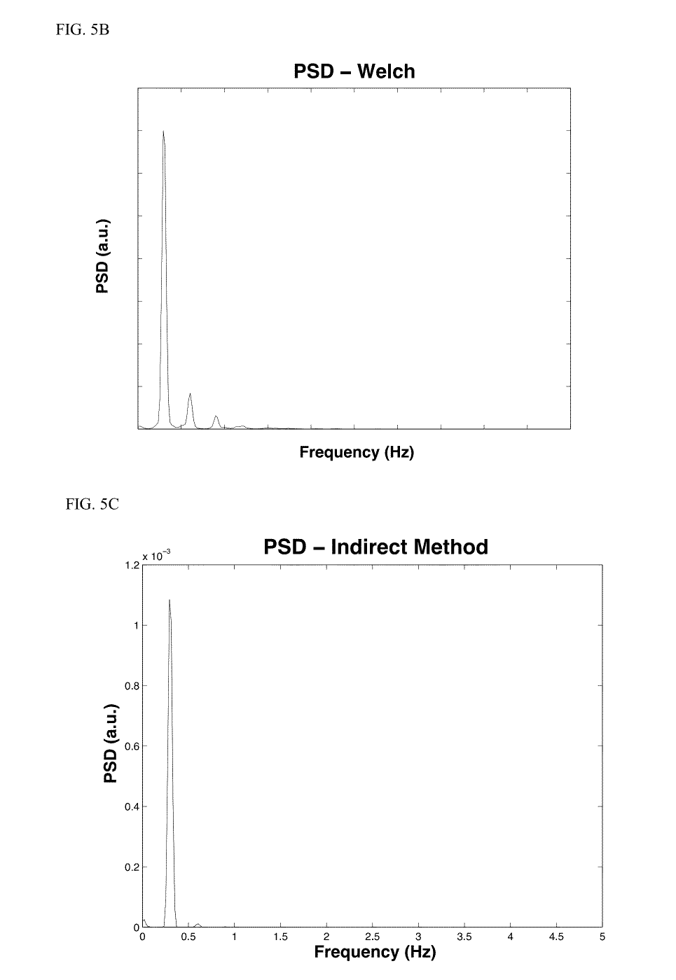

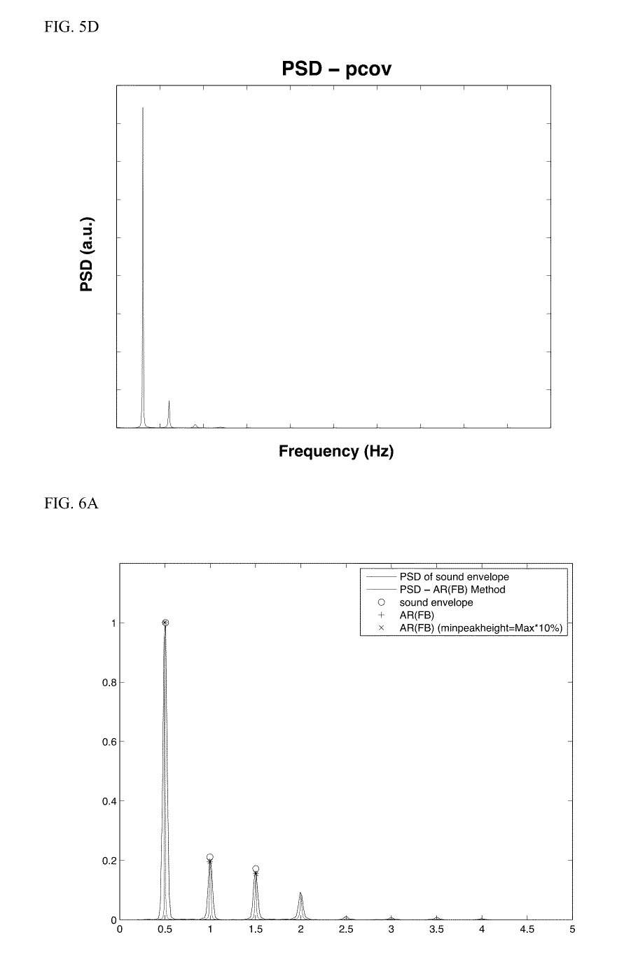

FIGS. 5A-5D illustrates sample graphs of a PSD using a sound envelope and an AR model.

Referring to FIGS. 5A-5D, two different samples of the PSD using an AR model of a general experimental subject and a sound envelope for the experimental subject suffering from nasal congestion are shown when the breathing rate is 0.3 Hz. The maximum peaks are obtained at a first-order frequency and a second-order frequency. In general, the breathing frequency may be determined to be a frequency corresponding to the maximum peak of the PSD.

The amplitude value (FIGS. 4A-4D) extracted from a flow that is estimated form the raw data is also represented together with the maximum peak of the PSD obtained at 0.3 Hz that is equal to the breathing frequency. However, as shown in FIGS. 5A-5D, the breathing rate of the experimental subject who suffered from nasal congestion was twice as high as in other cases.

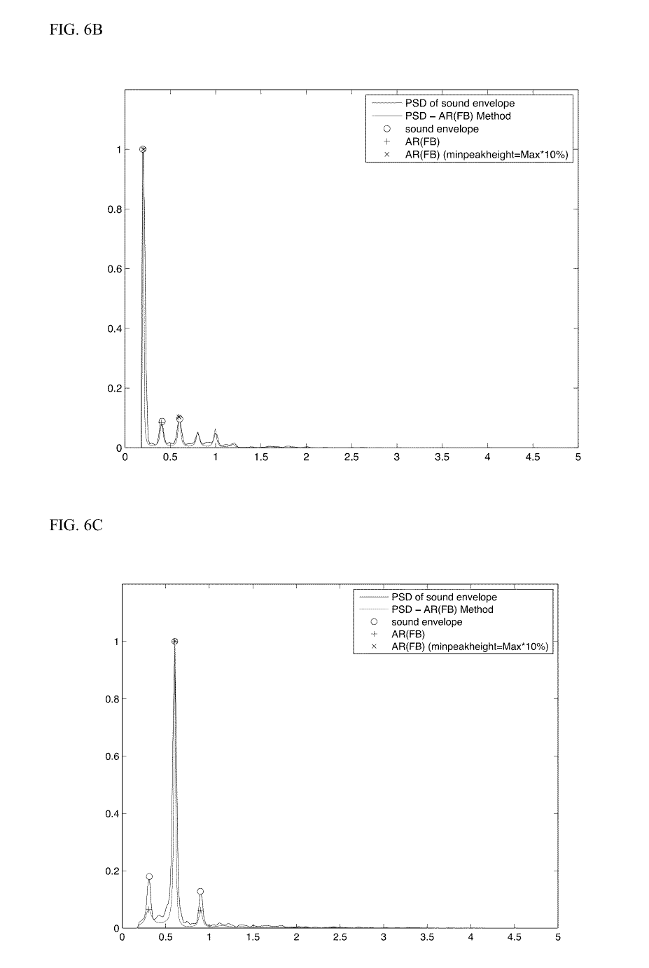

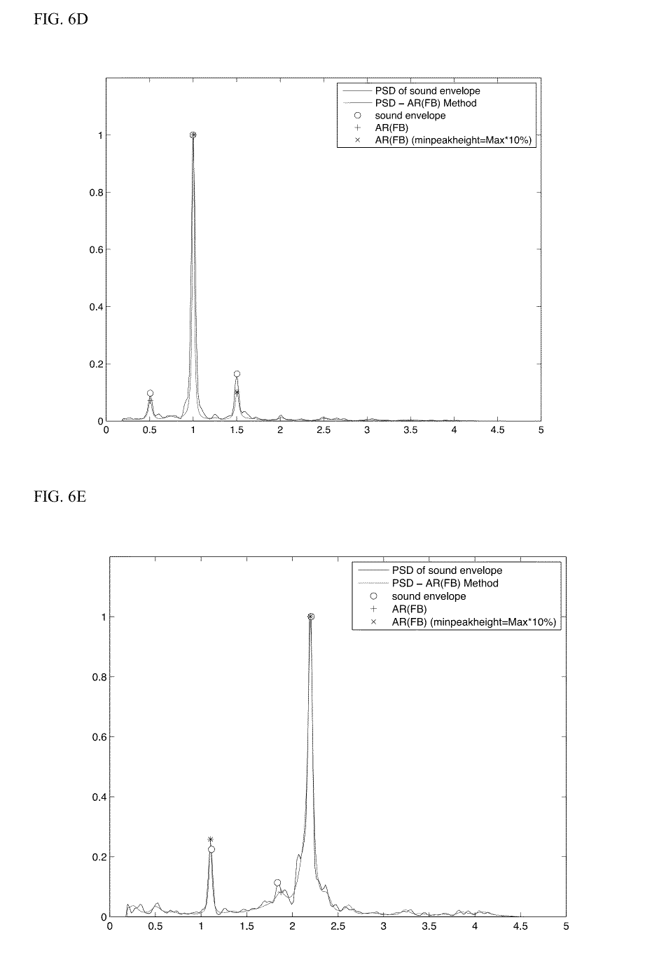

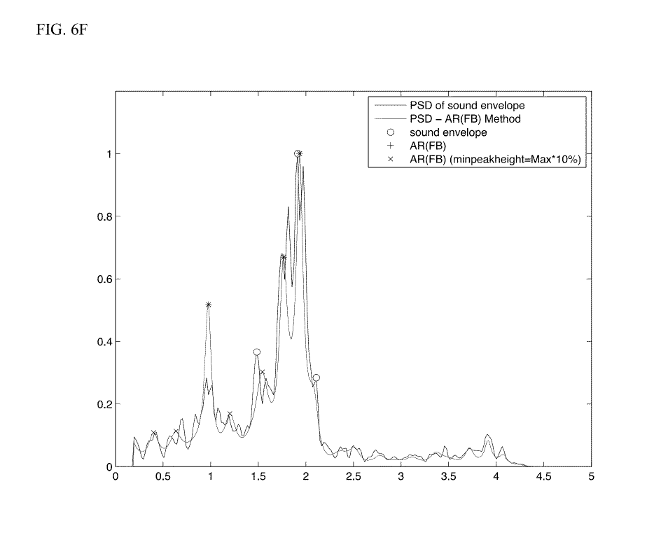

FIGS. 6A-6F illustrate peak graphs of a PSD using a sound envelope and an AR model.

Referring to FIGS. 6A-6F, different PSDs corresponding to nasal breathing activities of a total of 10 experimental subjects were obtained. It started at a peak indicative of a first cycle of a breathing sound signal after a flat region indicative of a second cycle. A shape was random, and a plurality of peaks were observed from the breathing sounds of respective experimental subjects. The blue and green solid lines indicate a PSD using an average and AR model of sounds of 10 experimental subjects, which have circles and crosses corresponding to the three peaks. As shown in FIG. 6A, while two peaks in a frequency range (1 Hz and 1.5 Hz) are matched with amplitude modulation of a breathing rate at 0.5 Hz, a maximum signal at 0.5 Hz indicates the breathing rate (indication of harmonics). Likewise, as shown in FIG. 6B, while two peaks in a frequency range (0.4 Hz and 0.6 Hz) are matched with amplitude modulation of a breathing rate at 0.2 Hz, a maximum signal at 0.2 Hz indicates the breathing rate (indication of harmonics). In FIGS. 6C and 6D, the maximum peaks at 0.6 Hz and 1 Hz are breathing rates while there are two return indices A¹. In FIGS. 6E and 6F, it was observed that there were three return indices A¹.

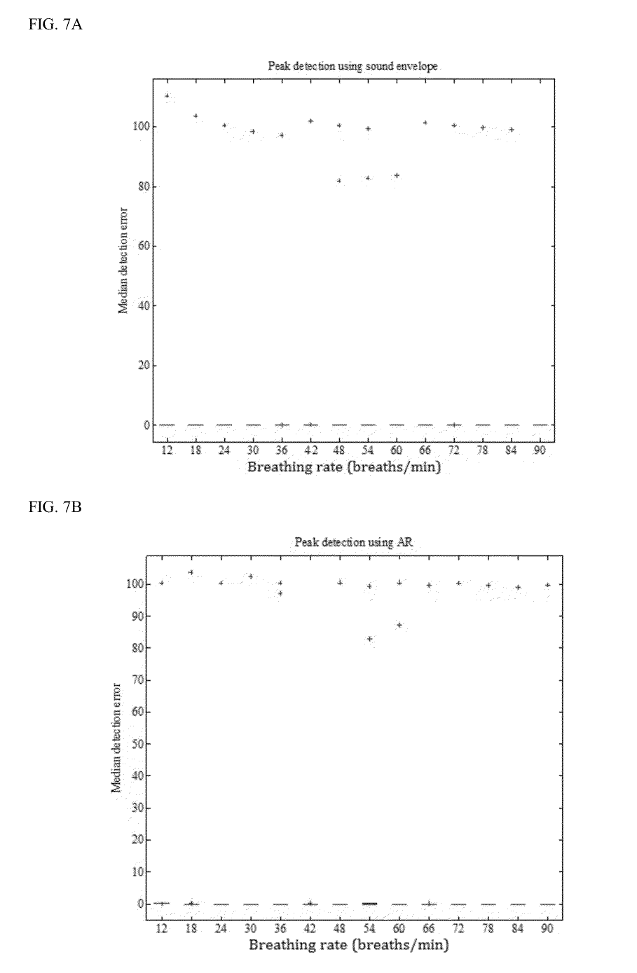

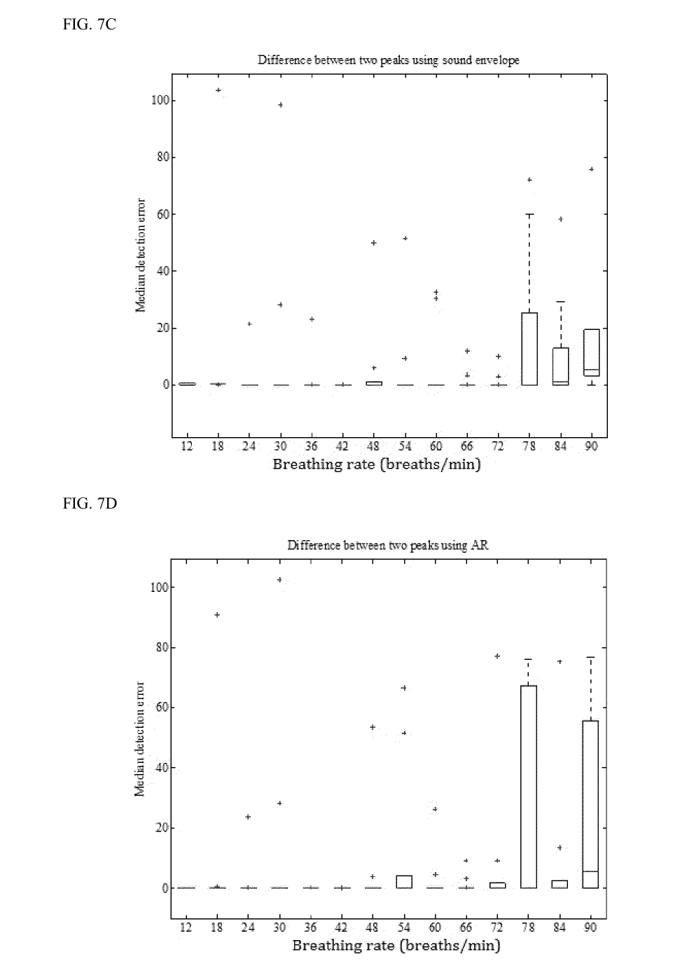

FIGS. 7A-7D illustrate graphs of median and IQR errors that are measured from a breathing rate result calculated by a maximum peak in a PSD using a sound envelope and an AR model.

Referring to FIGS. 7A-7D, as defined in Equation 8, the median and IQR errors are obtained from an actual breathing rate and a derived breathing rate, respectively. A bottom of a box, which is closest to zero, indicates a 25th percentile, a line in the box indicates a median, and a top of the box, which is farthest from zero indicates a 75th percentile. Whiskers in upper and lower portions of the box indicate 90th and 10th percentiles. Accordingly, a region of a blue box is an indication of diffusion according to the number of entities, that is, a change in the median (or IQR) error. This characteristic shows well that an algorithm is performed over an entire population. A red cross indicates 5th percentile and 95th percentile. In FIGS. 7A-7D, it can be seen that, except the 5th and 95th percentiles, ε of the sound envelope and the AR model are low in all breathing rates. ε of the sound envelope and the AR model are 10.44 and 13.39. Accordingly, there is no great difference in an average breathing rate estimation error between approaches that uses the maximum peak.

In order to reduce a median detection error in the 5th and 95th percentiles, a maximum peak and a second maximum peak are considered in a power spectrum of breathing data. The breathing rate is measured through a simple evaluation by an Euclidean distance between the maximum peak and the second maximum peak of the PSD. As shown in FIGS. 7A-7D, ε of the sound envelope and the AR model are 6.83 and 7.87. A difference in a median value that is defined by ε (the average breathing estimation error defined in Equation 8) is significantly low for an approach that is based on an Euclidean distance between the maximum peak and the second maximum peak.

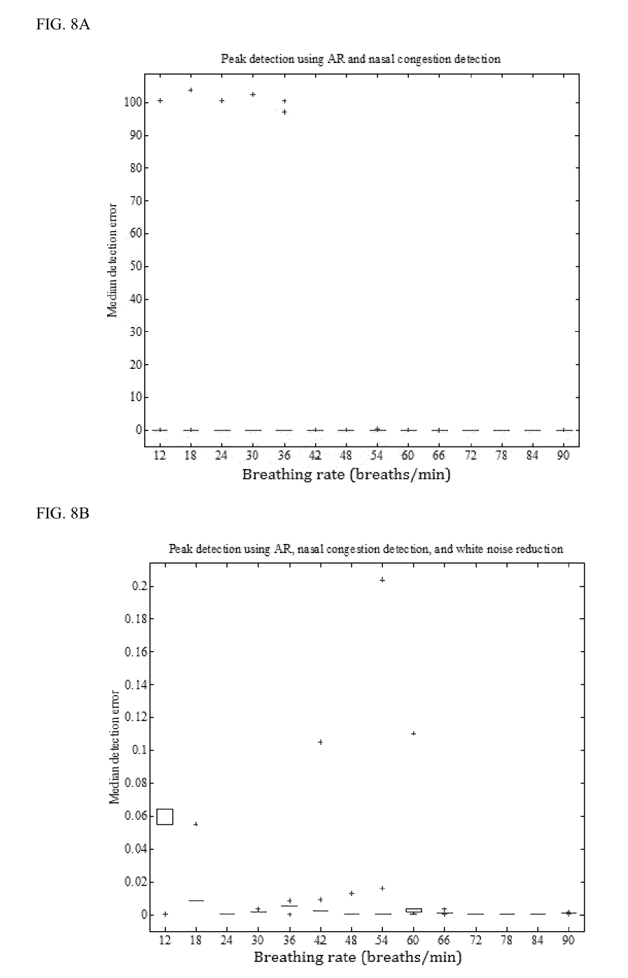

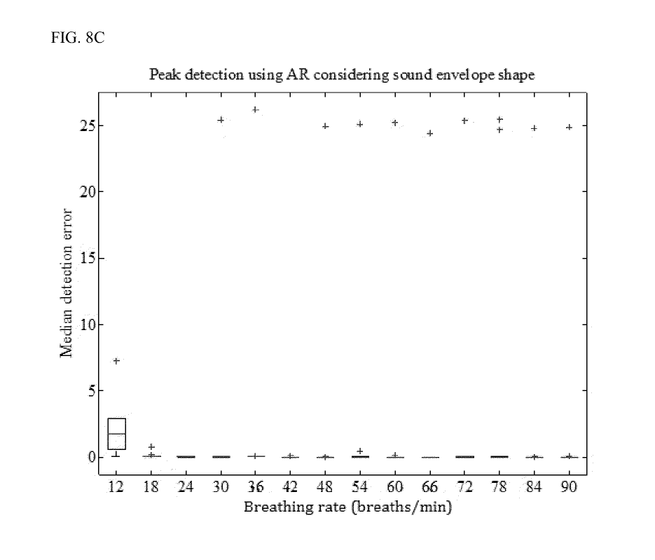

FIGS. 8A-8C illustrate graphs of median and IQR errors that are measured from a breathing rate result calculated by a maximum peak or by the maximum peak and a second maximum peak in a PSD using a sound envelope and an AR model.

As shown in FIG. 8A, ε of an AR model considering detection of nasal congestion is 5.04. An accuracy of estimation of the breathing rate in consideration of the detection of the nasal congestion is a little better than that in the above-mentioned approaches. In FIG. 8A, a median detection error of an LF breathing rate that is measured at 5th and 95th percentiles was caused by a white noise. As shown in FIG. 8B, ε of an AR model considering detection of that nasal congestion and detection of the white noise is 0.01. Table 1 summarizes measurement of a median error and an IQR error in a breathing rate between 0.2 Hz and 1.5 Hz. As provided as numerical numbers in Table 1, it can be seen that the AR model considering the detection of the nasal congestion and the detection of the white noise may provide and accommodate the lowest median error in all breathing rates. As shown in FIG. 8C, ε of the AR considering the shape of the sound envelope is 2.69.

As described above, several methods for estimating a breathing rate from a nasal breath sound signal are provided in an embodiment of the present invention. A smartphone was tested for feasibility to estimate a breathing rate using a microphone. The motivation of the present invention based on several previous studies is that a breathing rate, in particular, LF and HF breathing rates may be accurately obtained by a pulse oximeter. That is, a characteristic of a breath sound obtained from a microphone of a smartphone accurately matches the breathing rate. Thus, it is theoretically possible to obtain the accurate breathing rate. This result shows that, in the LF and HF breathing range of 0.2 to 1.5 Hz, the accurate breathing rate can be achieved from the breathing sound recorded from the microphone of the smartphone.

The sound envelope and the AR model were compared using a peak in a PSD of the tracheal and nasal sound signals with respect to the estimation of the breathing rate in the smartphone. In the present invention, all used methods provided accurate breathing estimation for the LF and HF breathing rates. In particular, the AR model considering the detection of the nasal congestion and reduction of the white noise provides the lowest median error in all breathing rates. For HF breathing rates (0.8 to 1.5 Hz), a simple estimation method for detecting a peak in a PSD cannot provide a good result because the experimental subjects suffer from nasal congestion caused by a cold or rhinitis that is naturally acquired.

A microphone's sensitivity is measured as a sine wave of 1 kHz (dB) at a sound pressure level (SPL) of 94 dB or as a pressure of 1 Pascal (PA). A magnitude of an analog or digital output signal from a microphone having the input stimulation is a sensitivity magnitude of the microphone. In the present invention, a sound signal was obtained by a smartphone having two microphones including an Infineon 1014 microphone positioned in an upper portion of the apparatus and a Knowles S1950 microphone positioned in a lower portion. The Infineon 1014 microphone was used to remove a background noise that is positioned in an upper portion of a unit around a headphone jack. A main microphone is positioned on the left of the bottom. Currently, smartphone OS devices (for example, iPhone 3GS and later, iPod touch 4 and later, and all iPads) include a built-in microphone. However, the products of Apple may include a very steep high-pass filter (low-frequency blocking) as a wind and pop filter. A low-frequency roll-off for the built-in microphone of the apparatus starts at 250 Hz and is very steep in the order of 24 dB/octave. However, with the advent of a smartphone OS6, a low-frequency roll-off filter may be released by a result of a significantly flat response. Though the performance of the smartphone was limited, these microphones were compensated as far as possible.

In analysis of the breathing sound, a better performance for detecting an apnoea-hypopnoea index (AHI) or a sleep apnoea/hypopnoea syndrome (SAHS) is recorded by the microphone of the smartphone that may be acquired when combined with a signal of an oximeter. In the present invention, spectrum morphology of the nasal sound signal is analyzed to develop a breathing rate estimation method. The change in intensity of the nasal sound signal was examined to select an optimal model that indicates such a relation to an evaluation flow.

Since persons may feel uncomfortable when using an earpiece microphone, although the smartphone is disposed on a table or hand without the earpiece microphone, non-contact breathing sound was acquired to describe that the breathing rate may be accurately induced from an audio signal that is obtained by a smartphone.

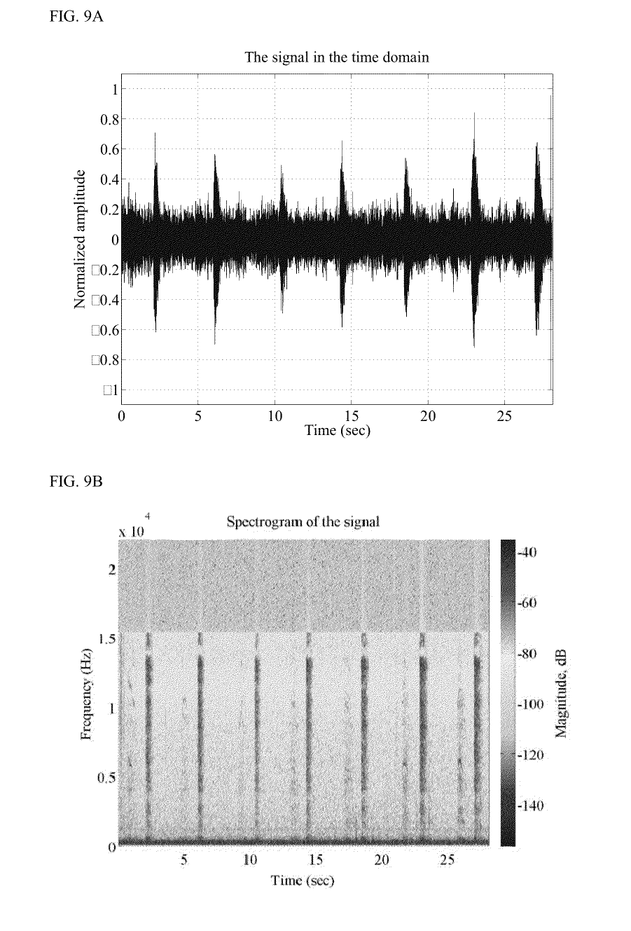

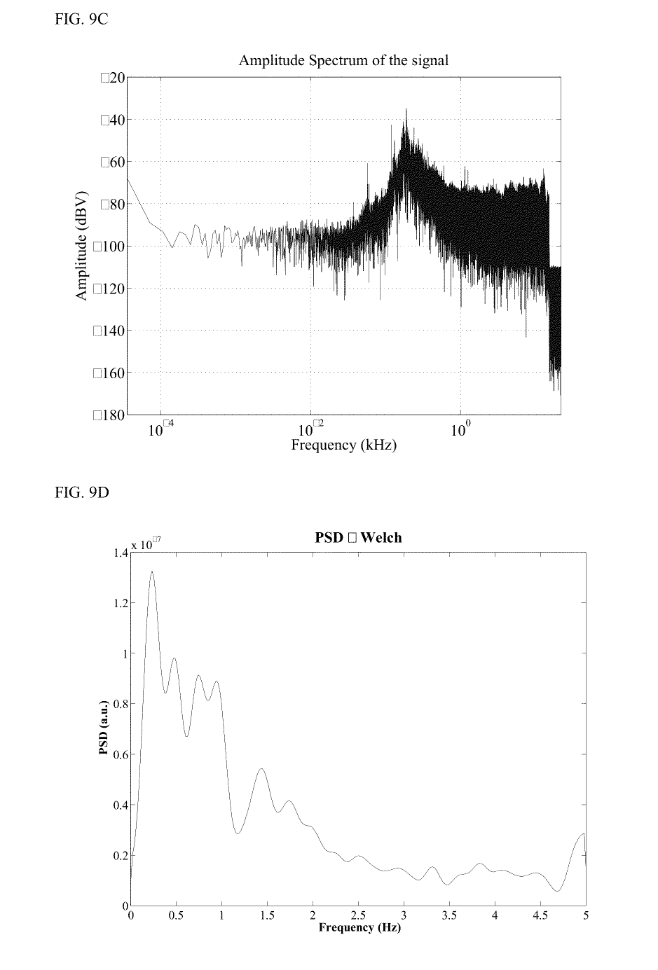

FIGS. 9A-9D illustrate graphs showing a typical nasal sound signal, sound spectrum, amplitude spectrum, and PSD that are obtained by a microphone built in a smartphone.

FIGS. 9A-9D is a record for 30 seconds, and FIG. 9D shows a main peak and a second peak around 0.2344 Hz and 0.4883 Hz.

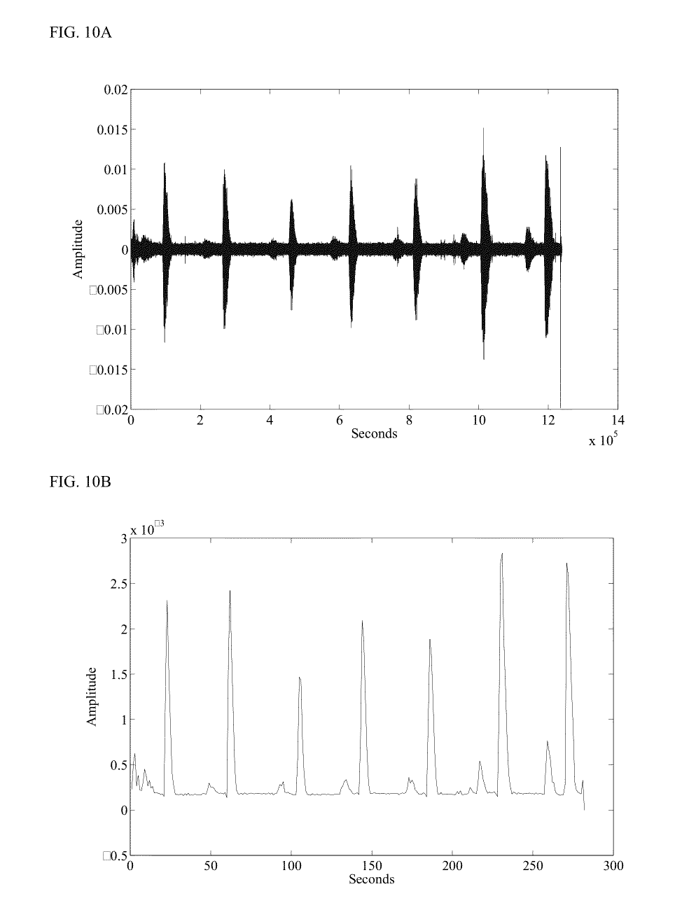

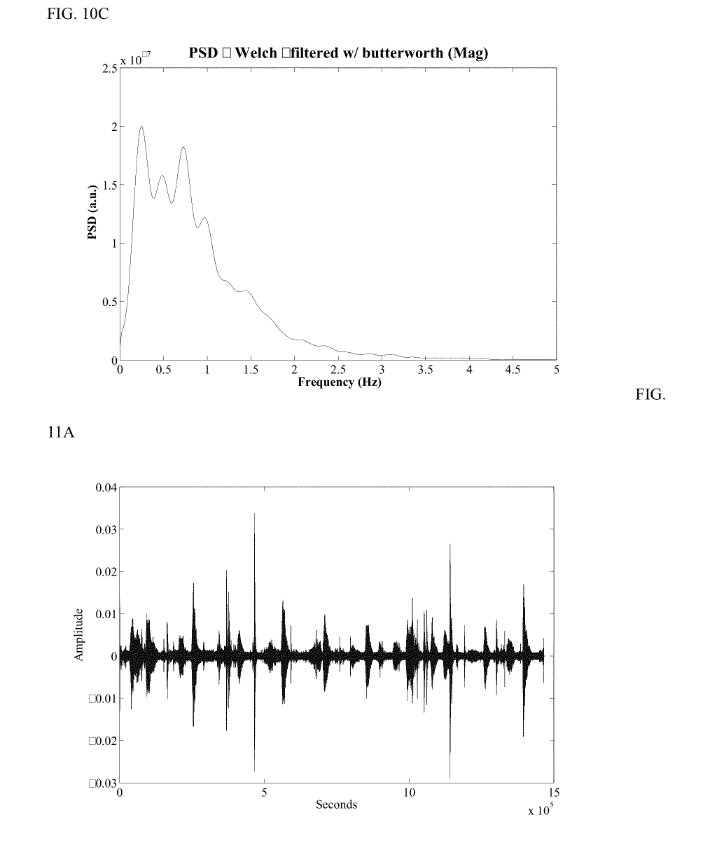

FIGS. 10A-10C illustrate raw data of a nasal breath sound that is recorded while breathing naturally when a distance between a nasal cavity and a smartphone is 30 cm.

Referring to FIGS. 10A-10C, there is a sample of the nasal breath sound that is recorded together with a background voice noise while breathing naturally when a distance between a nasal cavity and a smartphone is 30 cm. In FIGS. 10A and 10B, inhalation and exhalation were observed in a butterworth filter and a downsampled signal. FIG. 10C shows peaks around 0.2539 Hz, 0.4883 Hz, and 0.7227 Hz. An actual breathing rate is 0.2539 Hz.

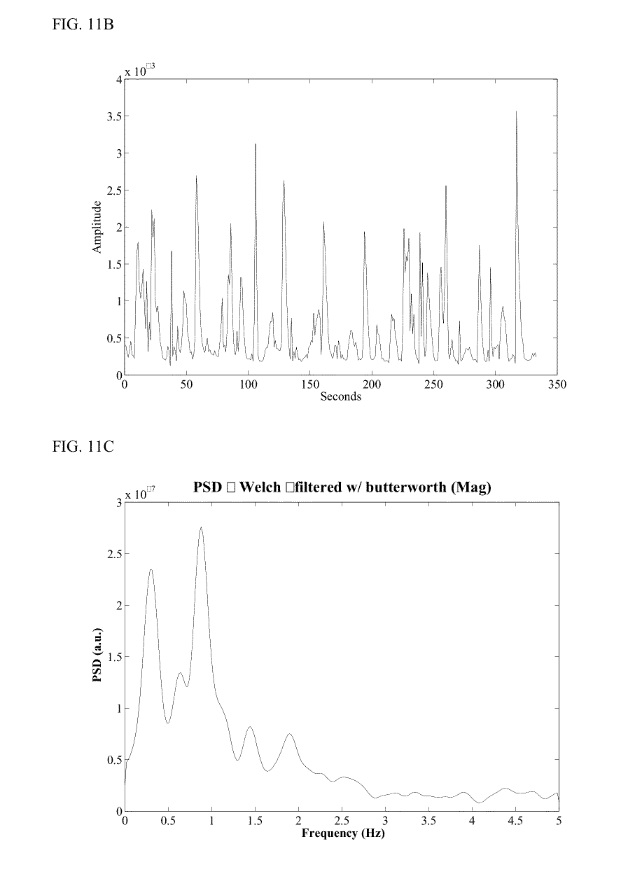

FIGS. 11A-11C illustrate a sample of a nasal breath sound that is recorded along with a background voice noise while breathing naturally when a distance between a nasal cavity and a smartphone is 30 cm.

In FIGS. 11A and 11B, the breathing rate was measured by a peak around 0.3125 Hz although there was often a background voice noise. An actual breathing rate is 0.3125 Hz. Even when the background noise includes a human voice, and a non-contact microphone is used, the estimation provided in the present invention provides reasonable good estimation of the breathing rate in the LF and HF breathing range. Through such an experiment, an additional linkage with another critical life sign function may be established from audio signals acquired from a smartphone or tablet.

As described above, with the apparatus and method for estimating the breathing rate using the microphone according to an embodiment of the present invention, it is possible to accurately estimate the breathing rate because calculation is performed in consideration of a dynamic characteristic of inhalation and exhalation, nasal congestion, and white noise.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

What is claimed is:

1. An apparatus for estimating a breathing rate using a microphone, the apparatus comprising:
a preprocessing unit configured to perform band filtering and noise filtering on tracheal and nasal sounds collected from the microphone;
a data selection unit configured to select a processing region of preprocessed data;
a similarity calculation unit configured to calculate similarity between pieces of data using an autocorrelation function;
a power spectrum calculation unit configured to calculate a power spectrum density;
a peak detection unit configured to detect multiple peaks including a highest peak through the power spectrum density;
a pattern determination unit configured to analyze the multiple peaks to determine a breathing pattern; and
a breathing rate calculation unit configured to calculate a breathing rate in consideration of a dynamic characteristic between inhalation and exhalation, nasal congestion detection, and noise reduction.

2. The apparatus of claim 1, wherein the breathing rate calculation unit comprises:
a nasal congestion detection unit configured to detect nasal congestion; and
a noise detection unit configured to detect background and voice noises.

3. A method of estimating a breathing rate using a microphone, the method comprising:
performing band filtering and noise filtering on tracheal and nasal sounds collected from the microphone;
selecting a processing region of preprocessed data;
calculating similarity between pieces of data using an autocorrelation function;
calculating a power spectrum density;
detecting multiple peaks including a highest peak through the power spectrum density;
analyzing the multiple peaks to determine a breathing pattern; and
calculating a breathing rate in consideration of a dynamic characteristic between inhalation and exhalation, nasal congestion detection, and noise reduction.

4. The method of claim 3, wherein the calculation of the breathing rate is performed using any one of a Welch periodogram method, an AR power spectrum (Burg algorithm), and a modified covariance method.
